(19) United States
(12) Patent Application Publication
(10) Pub. No.: US 2008/0318859 A1
(43) Pub. Date: Dec. 25, 2008

(54) Methods for Tissue Engineering

(75) Inventors: Anthony P. Hollander, Bristol (GB); Wa'el Z. Kafienah, Bristol (GB)

Correspondence Address: Edwards Angell Palmer & Dodge LLP;Peter C. Lauro, Esq., P.O. Box 55874, Boston, MA, 02205, US

(73) Assignee: THE UNIVERSITY OF BRISTOL, Bristol (GB)

(21) Appl. No.: 11/994,758

(22) PCT Filed: Jul. 6, 2006
(86) PCT No.: PCT/GB2006/002521

§ 371 Date: Jun. 27, 2008

(30) Foreign Application Priority Data
Jul. 6, 2005 (GB) 0513777.3

(51) Int. Cl. A61K 38/29; C12N 5/06; A01N 1/02; C07K 14/635

(52) U.S. Cl. 514/12; 435/325; 435/395; 435/1.1; 530/399

(57) ABSTRACT

The invention relates to the use of parathyroid hormone-related protein (PTHrP) in the prevention of hypertrophy in chondrogenic cells for cartilage replacement. A method for engineering three dimensional cartilage constructs from chondrogenic cells is provided, said method comprising a step of treating the chondrogenic cells or immature constructs with PTHrP to regulate hypertrophy. Also provided are: three dimensional cartilage produced by the method of the invention, and an engineered cartilage construct comprising chondrogenic cells and a bioactive scaffold capable of controlled release of PTHrP. In addition, the invention provides a method for the treatment of osteoarthritis.

This invention is in the field of tissue engineering. In particular, the invention relates to methods for use in cartilage tissue engineering and repair. The methods of the invention may be applied in the treatment of injuries or diseases which cause damage or degeneration of articular cartilage.

An intact articular cartilage surface is essential for normal joint function (1). Loss of this tissue through degradation of the type II collagen and proteoglycan components of its extracellular matrix is a well-described feature of osteoarthritis (OA) (1-4). In adults there is little or no capacity for self-repair of eroded articular cartilage, presumably because it is avascular (5, 6). Despite intensive research into the use of proteinase inhibitors to prevent cartilage loss in OA (7), no effective pharmaceutical therapies have emerged (8, 9). In recent years, a range of methods has been developed for the repair of articular cartilage lesions (5, 10). These include osteochondral transplantation (11), microfracture (6) and autologous chondrocyte implantation (ACI) (12, 13) with or without the assistance of a scaffold matrix to deliver the cells (14). A feature of all of these techniques is that their use is limited to the repair of focal lesions and patients with OA are mostly excluded from treatment. OA cartilage lesions are generally large and unconfined (15) and so do not provide an appropriate environment for chondrocytes or stem cells to be retained long enough to elaborate an extracellular matrix. Therefore successful repair of OA cartilage lesions is only likely to be achieved when three-dimensional cartilage implants can be generated that have enough extracellular matrix for fixation within the joint.

Cartilage tissue engineering provides a potential method for the production of three dimensional implants (16, 17). Effective engineering protocols have already been developed in which chondrocytes, usually from young animals, are seeded onto biodegradable scaffolds and cultured in a bioreactor (18, 19). Generating three-dimensional cartilage using adult human chondrocytes is far more challenging and in the case of older OA patients, is probably impossible in the clinical setting, because of the lack of autologous donor tissue. This has led a number of groups to explore the use of mesenchymal stem cells for the generation of autologous chondrocytes (20). These are mulitpotent cells with self-renewing capacity (21, 22). Many studies have utilised adherent bone marrow stromal cells (BMSCs) cultured as small micromass pellets and stimulated with TGF-β to drive chondrogenesis (23, 24). From these studies there is good histological evidence that under these conditions the BMSCs become chondrocytes and synthesize both type II collagen and protoeglycan. However micromass pellets were designed for use as an experimental model and the amount of extracellular matrix they produce is too small to be of practical value for implantation (25). Furthermore, there is clear evidence that BMSCs stimulated with TGF-β express type X collagen, an early marker of hypertrophy that is normally absent from hyaline cartilage (26). Finally, it is not yet known if BMSCs derived from OA patients (OA BMSCs) have the capacity to become chondrocytes and generate hyaline cartilage. Most studies have utilized BMSCs from animals or normal human donors (22-24, 27). However one study (28) investigated OA BMSCs cultured as pellets and concluded that they had a reduced chondrogenic capacity.

Longitudinal growth of long bones, ribs, and vertebrae is regulated by chondrocyte proliferation, matrix production, and a series of differentiation events in the fetal and juvenile growth plate (for review see (45)). Slowly proliferating chondrocytes in the resting zone of the epiphysis accelerate their cell cycle and align in a columnar array in the proliferating zone. These chondrocytes increase their volume and start expressing parathyroid hormone (PTH)/PTH-related protein (PTHrP) receptor (PTHR-1), followed by Indian hedgehog (Ihh) in the prehypertrophic zone (46-48). With further differentiation to hypertrophic chondrocytes the cell volume increases 7- to 10-fold, and cells start expressing high levels of type X collagen (Col X) and alkaline phosphatase, followed by bone-typical proteins, such as osteopontin, osteocalcin, and Cbfal (49). The hypertrophic chondrocytes develop microvilli and shed matrix vesicles which serve as nucleation centers for cartilage calcification (50). Finally, chondrocytes either become apoptotic (51) and are resorbed by chondroclasts in the course of cartilage resorption and replacement of bone or survive for some time in the calcified cartilage core of endochondral bone trabecules (52).

The major hormone systems controlling the proliferation and differentiation of chondrocytes in the growth plate are BMPs, bFGFs, as well as PTHrP and its receptor PTHR-1 (45). Available evidence suggests that PTHrP supports chondrocyte proliferation (53,54) and suppresses their differentiation to hypertrophic chondrocytes: PTHrP^{-/-} as well as PTHR-1^{-/-} mice show a chondrodysplastic phenotype owing to reduced proliferation and premature hypertrophy of growth plate chondrocytes (55){Kronenberg, 1998 #88}. In contrast, in mice overexpressing PTHrP (46,56) and in patients with a constitutively activated PTHR-1 (57), chondrocyte hypertrophy and subsequent endochondral ossification are delayed, again resulting in growth abnormalities. In the early embryonic epiphysis of rodents and chicken PTHrP is expressed predominantly in the perichondrium and periosteum (48). Synthesis of PTHrP is inhibited by BMP-2, -4, -6, and -7 (58) and stimulated by Ihh (48). However, the mechanism of control of chondrocyte proliferation is not fully understood and the precise role of PTHrP within the control system remains to be elucidated.

Stem cells are present throughout embryonic development as well as in several organs of the adult (59). They constitute a pool of undifferentiated cells with the remarkable ability to perpetuate through self-renewal whilst also retaining the potential to terminally-differentiate into various mature cell types (60). Bone marrow stromal cells. (BMSCs) can be easily isolated from adult marrow and contain a population of pluripotent progenitors that can give rise to mesenchymal lineages including chondrocytes, osteoblasts, fibroblasts and adipocytes (60). It is probable, however, that true mesenchymal stem cells represent a rare subpopulation of BMSCs (61-63). These cells are capable of dividing many times whilst retaining their ability to differentiate into various lineages with more restricted developmental potentials (64).

A growing area in regenerative medicine is the application of stem cells in cartilage tissue engineering and reconstructive surgery. This requires well-defined and efficient protocols for directing the differentiation of stem cells into the chondrogenic lineage. The use of exogenous cytokines and growth factors is a step forward in the development of a defined culture milieu for directing the chondrogenic differentiation of stem cells. Because the process of chondrogenesis is so closely intertwined with osteogenesis, many of the cytokines and growth factors that promote chondrogenic differentiation are also implicated in osteogenic differentiation (65,66). Hence, the challenge is to find an optimized subtle combination of these various cytokines and growth factors that would bias differentiation specifically toward the chondrogenic lineage.

One major problem of current cartilage repair techniques is that three-dimensional encapsulated mesenchymal progenitor cells frequently differentiate into hypertrophic cells that express type X collagen and osteogenic marker genes (67,68). It is therefore an object of the present invention to provide a method to inhibit the hypertrophy of stem cells in chondrogenic, three-dimensional cultures.

The present invention arises from the inventors' observation that PTHrP can inhibit type X collagen, a marker of hypertrophy, in chondrogenic 3D cultures of BMSCs.

Accordingly, in a first aspect, the invention provides the use of PTHrP in the prevention of hypertrophy in chondrogenic cells for cartilage replacement. As used herein, “PTHrP” means PTHrP and any homologue, analogue, derivative or fragment thereof, natural or synthetic, irrespective of its source, which retains the ability of PTHrP to inhibit hypertropyhy in chondrogenic cells. Preferably, the PTHrP homologue, analogue, derivative or fragment retains the ability to interact with the PTHrP receptor PTHR-1.

As used herein, “chondrogenic cells” means any cells capable of giving rise to or forming cartilage including, but not limited to: stem cells (e.g. bone marrow stromal cells, umbilical cord blood stem cells, embryonic stem cells) and chondrocytes.

In a preferred embodiment, the chondrogenic cells are bone marrow stromal cells (BMSCs). The chondrogenic cells may be autologous (i.e. obtained from the patient) or non-autologous (i.e. obtained from a donor who is not the patient; also called allogeneic). It is predicted that a first application of the present invention will employ autologous BMSCs. The use of autologous cells has several advantages. It avoids the risk of immune rejection or the need for immunosuppression that would be required for donor cells. It also avoids the risk of disease transmission from donor to patient. In this respect, the generation of relatively mature cartilage implants using stem cells derived from the bone marrow of osteoarthritis patients described herein opens the possibility of developing a cartilage therapy for osteoarthritis utilising autologous stem cells. Previously, OA BMSCs have been reported to have a poor capacity to proliferate and form chondrocytes compared to normal BMSCs. The present invention succeeds in overcoming the reduced potential of OA-derived cells (although the application of the invention is not limited to OA-derived cells).

In a further aspect, the invention provides a method for preventing hypertrophy of chondrogenic cells in engineered cartilage tissue which comprises incubating the chondrogenic cells with PTHrP. In a preferred embodiment, the chondrogenic cells are chondrogenic BMSCs.

This aspect of the invention can alternatively be characterized as a method for engineering three dimensional hyaline cartilage from chondrogenic cells, which method comprises a step of treating the chondrogenic cells, or immature constructs, with PTHrP to regulate hypertrophy. The invention also provides three dimensional cartilage produced by said method.

Usually chondrogenic cells are seeded onto a scaffold or membrane support as known in the art. Any support known in the art may be used, for example the “cell bandage” described in WO 2006/032915.

The method may comprise incubation of PTHrP with chondrogenic BMSCs during the in vitro maturation of tissue engineered constructs before implantation.

Alternatively, the method may comprise administration of PTHrP to a patient following remedial surgery. In a preferred embodiment, the method comprises injection of PTHrP into the joint when using immature constructs seeded with BMSCs. However, in some cases systemic injection of PTHrP (iv/im) may be preferable. In addition, oral administration of PTHrP may be possible using a suitable synthetic variant.

A further possibility is the seeding of bioactive scaffolds that can slowly release PTHrP in situ following implantation. The scaffold or membrane may additionally comprise other factors for release such as TGF-β which is known to induce the production of chondrocytes from bone marrow cells. Accordingly, the invention also provides an engineered cartilage construct comprising chondrogenic cells and a bioactive scaffold capable of controlled release of PTHrP.

In another aspect, the invention provides a method for making pre-hypertrophic chondrocytes which comprises incubating BMSCs with PTHrP.

In a further aspect, the invention provides the use of PTHrP in the manufacture of a medicament for the regulation of hypertrophy in engineered cartilage.

Also provided is the use of PTHrP in the manufacture of engineered cartilage for the repair of damaged cartilage, in particular cartilage damage resulting from osteoarthritis.

In a still further aspect, the invention provides a method for the treatment of osteoarthritis which comprises administering to a patient in need thereof an effective amount of PTHrP, wherein hypertrophy of osteoarthritic chondrocytes is reversed or delayed.

The method may comprise:
the injection of PTHrP into osteoarthritic joints;
pre-treatment of osteoarthritic chondrocytes during expansion, before use in tissue engineered implants;
gene therapy of osteoarthritic chondrocytes with the PTHrP gene;
using pharmacological compounds that can directly upregulate the expression of PTHrP or its receptor.

In yet another aspect, the invention provides a method of screening compounds for PTHrP-like activity, i.e. the ability to inhibit hypertropyhy in chondrogenic cells, which comprises incubating a test compound with chondrogenic or chondrogenic progenitor cells and determining the production of type X collagen by the cells relative to control cells.

In a simple screen, the chondrogenic or chondrogenic progenitor cells may be bone marrow cells cultured on plastic.

Production of type X collagen may be determined by direct measurement of mRNA or through using a promoter-reporter construct.

In a secondary screen, the chondrogenic cells could comprise a cartilage engineering system, e.g. from bone marrow cells.

A suitable positive control in any such screen would be PTHrP itself.

Preferred embodiments of the different aspects of the invention are as to each other mutatis mutandis.

Embodiments of the invention are described in the following non-limiting example in which reference is made to the figures of which:

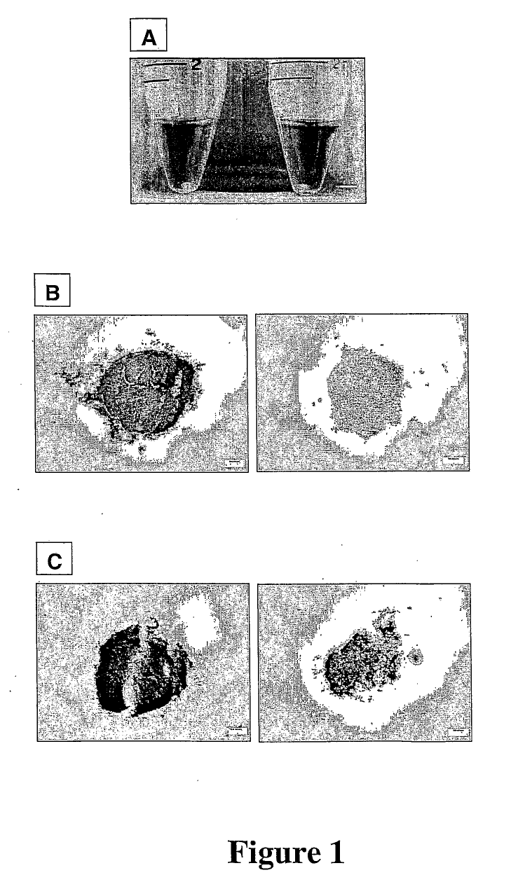

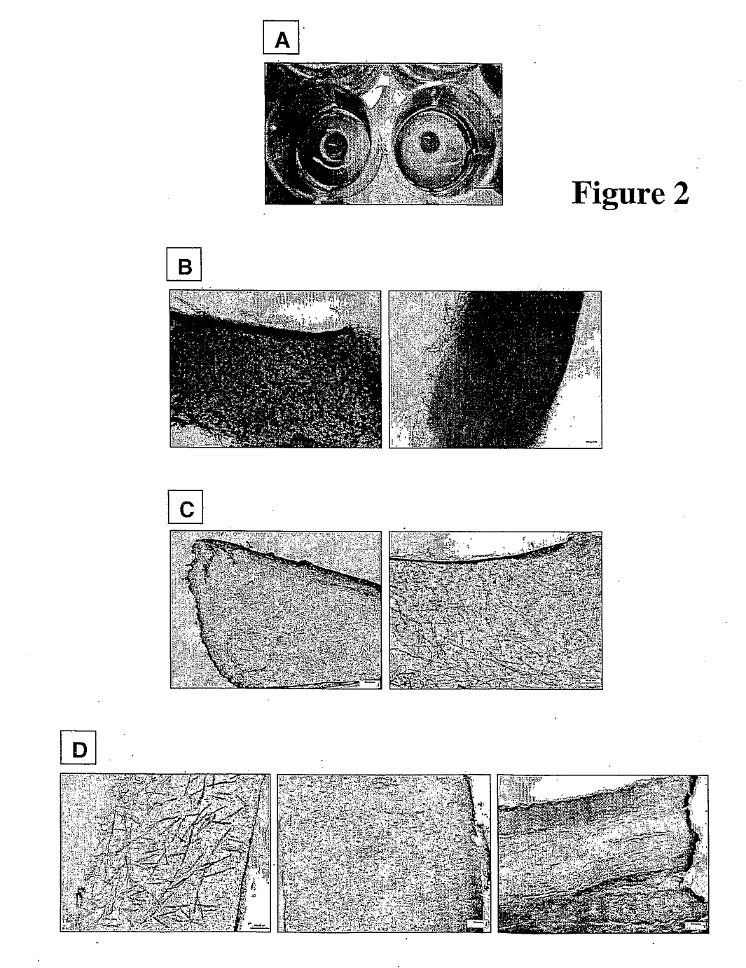

FIG. 1. Chondrogenesis in BMSC pellet cultures. Expanded OA BMSCs from passage 2 or 3 were cultured as three-dimensional pellets as described under Materials and Methods. A, Macroscopic appearance of pellets (scale bar is 3 mm). B-C, Histological appearance of pellets at the end of culture (scale bar is 100 nm). The sections were stained with haematoxylin and eosin (B, left panel), safranin O for sulfated proteoglycans (B, right panel) and for type II (C, left panel) and type I (C, right panel) collagens, using specific antibodies. The relevant positive and negative controls are shown in FIG. 2.

FIG. 2. Cartilage tissue engineering from BMSCs. Expanded OA BMSCs from passage 2 or 3 were used to engineer cartilage on PGA scaffolds, as described under Materials and Methods. A, Macroscopic appearance of engineered cartilage (scale bar is 3 mm). B-C, Histological appearance of engineered cartilage at the end of culture (scale bar is 100 nm). The sections were stained with haematoxylin and eosin (B, left panel), safranin O for sulfated proteoglycans (B, right panel) and immunostained for type II collagen (C, left panel) and type I collagen (C, left panel), using specific antibodies. D, Controls for immunostaining. The left panel is the negative control (normal goat serum), showing staining of the remaining PGA scaffold but not the extracellular matrix. Positive controls are shown for type II collagen in hyaline cartilage (middle panel) and type I collagen in tendon (right panel).

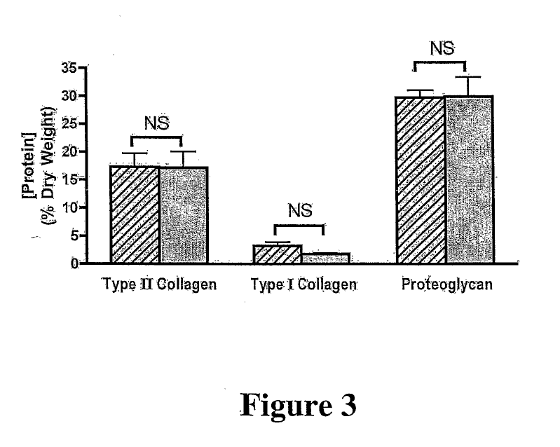

FIG. 3. Quantitative comparison of cartilage engineered from chondrocytes and BMSCs. Cartilage was engineered from bovine nasal chondrocytes (striped bars; n=18) or from expanded osteoarthritic BMSCs at passage 2 or 3 (grey bars; n=19) and then digested with trypsin, as described under Materials and Methods. The digests were assayed for collagen types I and II using specific immunoassays. Proteoglycan was measured as sulfated glycosaminoglycans using the dimethylmethylene blue colorimetric assay. The content of each protein is expressed as a % of dry weight and the results are shown as Mean±SEM. For each protein, the nasal chondrocytes and BMSCs were compared using the 2-tailed Mann-Whitney U-test. NS=not significant.

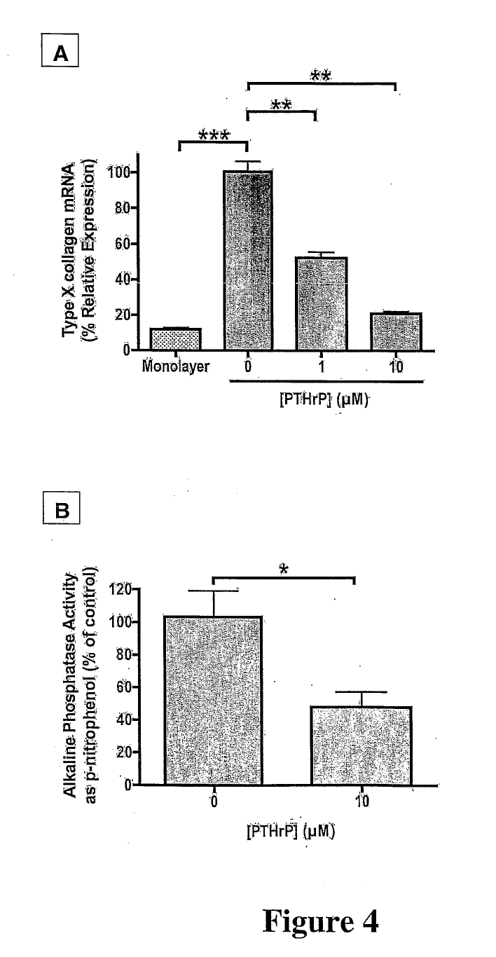

FIG. 4. Inhibition of hypertrophy by PTHrP. Expanded osteoarthritic BMSCs from passage 2 or 3 were cultured in monolayer (stippled bar) or used to engineer cartilage on PGA scaffolds with and without PTHrP (grey bars), as described under Materials and Methods. A, Analysis of type X collagen mRNA by quantitative real time PCR at the end of culture. Results have been normalized to the TGF-β control (0 PTHrP) and are shown as the Mean±SEM for 7 patients; **p<0.01, ***p<0.0001; 2-tailed Mann-Whitney U test with a Dunn's post-hoc correction. B, Alkaline phosphatase content determined by reaction with p-nitrophenyl phosphate as described under Materials and Methods; the enzyme activity is normalized to the control group (no PTHrP). Results are shown as the Mean±SEM for 6 patients. *p<0.05; 2-tailed Mann-Whitney U test.

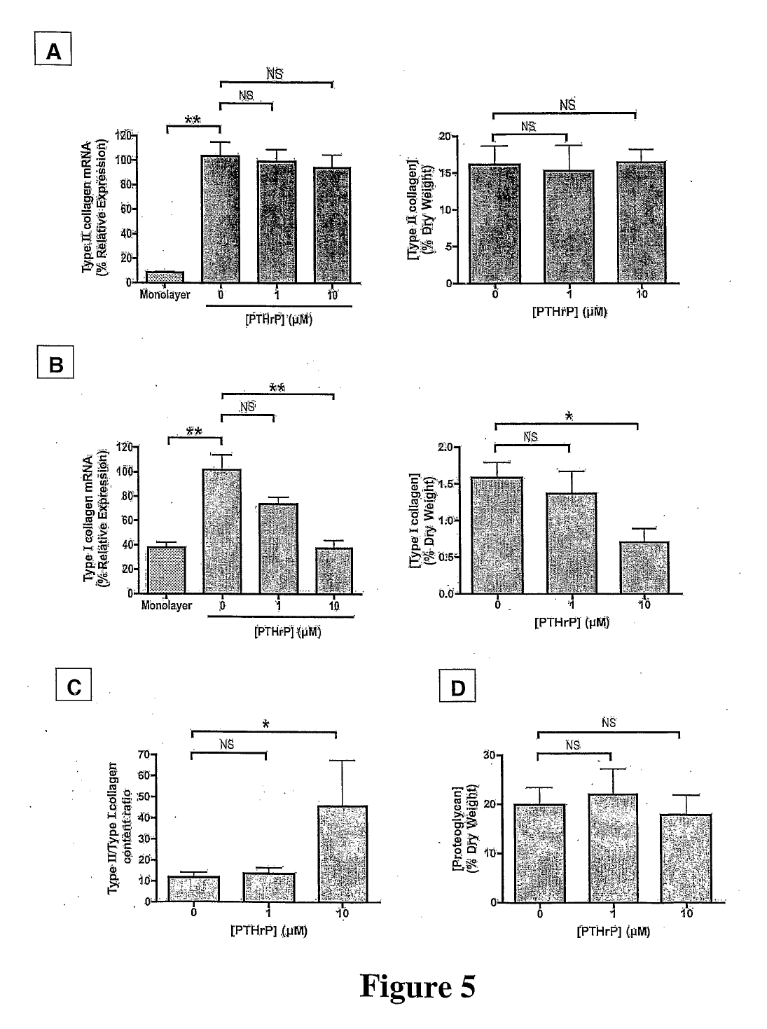

FIG. 5. Effect of PTHrP on the extracellular matrix of engineered cartilage. Expanded osteoarthritic BMSCs from passage 2 or 3 were cultured in monolayer (stippled bar) or used to engineer cartilage on PGA scaffolds with and without PTHrP (grey bars), as described under Materials and Methods. Collagen types II (A) and I (B) were analysed by quantitative real time PCR for mRNA (left panel; n=6 in each case) and by specific immunoassay of trypsin digests for protein (right panel; n=7 in each case). The results for mRNA have been normalized to the TGF-β control (0 PTHrP). C, Ratio of collagen II/collagen I measured as protein (n=7 in each case). D, Proteoglycan, measured as sulfated glycosaminoglycans using the dimethylmethylene blue colorimetric assay (n=7 in each case). All results are shown as the Mean±SEM. *p<0.05, **p<0.01, NS=Not Significant; 2-tailed Mann-Whitney U test with a Dunn's post-hoc correction.

### EXAMPLE

### Materials and Methods

### Patients

Bone marrow plugs were collected from the femoral heads of 23 OA patients undergoing hip arthroplasty at Southmead Hospital, Bristol of which 52% were male and 48% female. Their mean age was 65.8 years (range 42-90 years). The study was carried out in full accordance with local ethical guidelines and all the patients gave their informed consent.

### Isolation and Characterization of BMSCs

Cells were isolated from the bone marrow plugs by washing in expansion medium consisting of low glucose Dulbecco's Modified Eagles Medium (DMEM; Sigma) supplemented with 10% Foetal Bovine Serum (FBS), 1% (v/v) Glutamax (1×; Invitrogen) and 1% (v/v) Penicillin (100 U/ml)/Streptomycin (100 μg/ml) (Invitrogen). The serum batch was selected to promote the growth and differentiation of mesenchymal stem cells (29). The cell suspension was separated from any bone in the sample using a 19-guage needle. The cells were centrifuged at 1500 rpm for 5 minutes and the supernatant/fat removed. The resulting cell pellet was resuspended in medium, and then plated at a seeding density of between 1.5-2.0×10⁵ nucleated cells per cm². The medium was supplemented also with 1 ng/ml FGF-2 (Peprotech UK) to enhance BMSC proliferation and differentiation (30, 31). These flasks were incubated at 37° C. in a humidified atmosphere of 5% CO₂ and 95% air. The first medium change was after four days and then the medium was changed every other day until adherent cells reached 90% confluence and were ready for passaging. The cells were characterised for stem cell surface markers and multilineage potential as described previously (29).

### Micromass Pellet Cultures Using OA BMSCs.

Expanded BMSCs were trypsinized and cultured in micromass pellets as described previously (23) with slight modification. Briefly, 500,000 cells were placed in a 15-ml conical polypropylene tube and resuspended in 0.5-ml chondrogenic differentiation medium consisting of DMEM containing 4.5 g/l glucose supplemented with 10 ng/ml of transforming growth factor-3 (TGF-_{—}3; R&D Systems), 1 mM sodium pyruvate (Sigma), 50 μg/ml ascorbic acid-2-phosphate (Sigma), 1×10₋₇ M dexamethasone (Sigma), 1% ITS (Invitrogen), and 1% (v/v) Penicillin (100 U/ml)/Streptomycin (100 μg/ml) (Invitrogen). Cells were centrifuged at 1500 rpm for 5 minutes at 20° C. The pellets were maintained in culture with 1 pellet/tube and 0.5 ml chondrogenic medium/tube. Medium was changed every 2-3 days. After the first week the medium was further supplemented with 50 μg/ml insulin (Sigma) until the end of culture. Chondrogenic pellets were harvested at 21 days for mRNA, matrix proteins and histological analyses.

### Tissue Engineering Using OA BMSCs

PGA scaffolds (a kind gift from Dr. James Huckle and Dr. Andrew Jackson, Smith & Nephew, York, UK) were produced as 5 mm diameter×2 mm thick discs according to established method (32). The scaffolds were pre-soaked in 100 μg/ml human fibronectin (Sigma) in PBS to support BMSC adherence to PGA fibres. BMSCs from passage 2 or 3 were trypsinized and suspended in 30 μl of expansion medium. The suspension was loaded drop wise onto the scaffold in tissue culture wells pre-coated with 1% (w/v) agarose (Sigma) to prevent cell adherence to plastic. After incubation for 4 hours, the scaffolds were turned over and incubated for further 4 hours to allow for even distribution of cells across the scaffold. The constructs were maintained in a chondrogenic differentiation medium as described above for micromass pellet cultures. Human recombinant PTHrP (Peprotech) was included in the differentiation medium at 1 or 10 μM, where appropriate. The medium was changed three times a week. The constructs were incubated at 37° C., 5% CO₂ on a rotating platform at 50 rpm for 35 days. Harvested samples were digested with collagenase to release the cells, which were stored in −70° C. for subsequent RNA extraction or alkaline phosphatase activity assay. Other samples were stored in −20° C. prior to quantitative biochemical analysis (see below). In some experiments cartilage was engineered using bovine nasal chondrocytes that were isolated as described previously (33).

### Histological and Immunohistochemical Analysis of Micromass Pellets and Engineered Cartilage

Micromass pellets and mature cartilage engineered from stem cells was frozen in O.C.T. embedding matrix (BDH). Full-depth sections (thickness, 7 μm) were cut with a cryostat and fixed in 4% (w/v) paraformaldehyde (Sigma) in PBS, pH 7.6. Some sections were stained with haematoxylin and eosin (H&E) or 0.1% (w/v) safranin O (both from Sigma) to evaluate matrix and proteoglycan distribution, respectively. Other sections were immunostained with monoclonal antibodies against collagen types I and II (Southern Biotechnology), as previously described (33). Biotinylated secondary antibodies were detected with a peroxidase-labelled biotin-streptavidin complex (Vectastain Elite kit; Vector Laboratories, Peterborough, UK) with diaminobenzidine substrate (Vector Laboratories). Natural cartilage and tendon were used as positive controls for type II collagen and type I collagen, respectively. Normal goat serum was used as a negative control and all sections were counterstained with hematoxylin (Vector Laboratories).

### Quantitative Biochemical Analyses of Tissue-Engineered Cartilage

Dry weights of the constructs were determined after freeze-drying. The samples were then solubilised with trypsin and processed for complete biochemical analysis, as recently described (34). Briefly, samples were digested with 2 mg/ml TPCK-treated bovine pancreatic trypsin containing 1 mM iodoacetamide, 1 mM EDTA and 10 μg/ml pepstatin A (all from Sigma). An initial incubation for 15 h at 37° C. with 250 μl trypsin was followed by a further 2 h incubation at 65° C. after the addition of a further 250 μl of the freshly prepared proteinase. All samples were boiled for 15 min at the end of incubation, to destroy any remaining enzyme activity. The extracts were assayed by inhibition ELISA using a mouse IgG monoclonal antibody to denatured type II collagen, COL2-3/4m, as previously described (2). Peptide CB11B (CGKVGPSGAP—[OH]GEDGRP[OH]GPP[OH]GPQY) was synthesized using 9-fluorenyl-methoxycarbonyl chemistry, by Dr. A. Moir (Kreb's Institute, Sheffield University, UK) and was used as a standard in all of the immunoassays. The extracts were also assayed by inhibition ELISA using a rabbit antipeptide antibody to type I collagen, as previously described (34). Peptide (SFLPQPPQ) was synthesized using 9-fluorenyl-methoxycarbonyl chemistry, by Dr. A. Moir (Kreb's Institute, Sheffield University, UK) and was used as a standard in all of the immunoassays. Proteoglycan in the digests was measured as sulfated glycosaminoglycan by colorimetric assay using dimethylmethylene blue (Aldrich, Gillingham, UK) as previously described (35).

### Alkaline Phosphatase Activity

Cells in engineered cartilage constructs were assayed for alkaline phosphatase activity after collagenase-digestion of the extracellular matrix, as described previously (36). Briefly, the cells were lysed with 0.1 ml of 25 mM sodium carbonate (pH 10.3), 0.1% (v/v) Triton X-100. After 2 min each sample was treated with 0.2 ml of 15 mM p-nitrophenyl phosphate (di-tri salt, Sigma) in 250 mM sodium carbonate (pH 10.3), 1.5 mM MgCl₂. Lysates were then incubated at 37° C. for 2 h. After the incubation period, 0.1 ml aliquots were transferred to a 96-well microtitre plate and the absorbance read at 405 nm. An ascending series of p-nitrophenol (25-500 μM) prepared in the incubation buffer enabled quantification of product formation.

### RNA Isolation and Reverse Transcription

RNA was extracted from cell cultures using Sigma's GenElute™ Mammalian Total RNA kit, according to the manufacturers instructions. Reverse transcription (RT) was carried out using the Superscript II system (Invitrogen). Total RNA (2 μg) was reverse transcribed in a 20-μl reaction volume containing Superscript II (200 U), random primer (25 μM), dNTP (0.5 mM each) ay 42° C. for 50 minutes.

### Primer Design

The coding sequences for human type X, type II and type I collagens (Accession No. NM_{—}000493, NM_{—}001844 and NM_{—}000088, respectively) were used to design primers using the online software, Primer3 (Whitehead Institute for Biomedical Research, MIT). The primers span intronic junctions to avoid the amplification of genomic sequences. They were also checked for the amplification of potential pseudogenes. A BLAST search against all known sequences confirmed specificity. Published primers for the housekeeping gene β-actin (37) were used as a reference for normalization in all RT-PCR reactions. These primers had been specifically designed to not co-amplify processed pseudogenes in contaminating genomic DNA. All the primers generated the correct sizes of the PCR fragments with no nonspecific products, confirming the specificity of the real time RT-PCR (data not shown). Details of the primers used in the study are:

Collagen X α (F)
GACACAGTTCTTCATTCCCTACAC,
Collagen X α (R)
GCAACCCTGGCTCTCCTT,
Collagen II α1 (A + B) (F)
CAACACTGCCAACGTCCAGAT,
Collagen II α1 (A + B) (R)
CTGCTTCGTCCAGATAGGCAAT,
Collagen I α1 (F)
AGGGCCAAGACGAAGACATC,
Collagen I α1 (R)
CAACACTGCCAACGTCCAGAT,
β-Actin (F)
GACAGGATGCAGAAGGAGATTACT,
β-Actin (R)
TGATCCACATCTGCTGGAAGGT.

### Quantitative Real Time Reverse Transcriptase-Polymerase Chain Reaction

Quantitative real time polymerase chain reaction (PCR) was performed in a 25-μl reaction volume containing 12.5 μl of the SYBR Green PCR master mix (Sigma), 5 μl of the RT reaction mixture, and 300 nM primers using the Smart Cycler II System (Cepheid). For the β-actin gene, the RT reaction mixture was diluted 100 times. The amplification program consisted of initial denaturation of 95° C. (2 min) followed by 40 cycles of 95° C. (15 s), annealing at 58° C. (30 s), and extension at 72° C. (15 s). After amplification, melt analysis was performed by heating the reaction mixture from 60 to 95° C. at a rate of 0.2° C./s. The cycle threshold (Ct) value for each gene (X) of interest was measured for each RT sample. The Ct value for β-actin was used as an endogenous reference for normalization. The relative transcript level of a given gene (X) at a give treatment time point (T) over its value at the initial treatment time (0) was calculated as 2^{-ΔΔCt}, in which ΔCt=C_{tT}−Cₜ₀; C_{tT}=C_{tT,X}−C_{tT} and Cₜ₀=C_{t0,X}−C_{t0,β}. Real time RT-PCR assays were done in duplicate or triplicate and repeated two to four times.

### Statistical Analysis

Comparison of differences between individual groups was by the 2-tailed Mann-Whitney U test and p<0.05 was taken as significant. Where multiple comparisons were being made, the groups were compared by analysis of variance using the non-parametric Kruskal-Wallis test with p<0.05 taken as significant. Where significant variance was demonstrated, differences between individual groups were then determined using the two-tailed Mann-Whitney U-test with a Dunn's post hoc correction. p<0.05 was taken as significant.

### Results

### Phenotype of Isolated OA BMSCs

A well-characterised population of stem cells was used for the three-dimensional engineering of cartilage tissue. We have previously described this population as being positive for CD105, CD49a, CD117, BMPR-1A, STRO-1 and VCAM-1A and negative for CD34 (29). We also showed the population to be multipotential as it consistently differentiated into adipogenic, chondrogenic and osteogenic lineages (29). In the present study there was no significant variation in the extent of differentiation among the patient samples (n=23), between male and female patients, or with age (not shown), indicating again that the BMSC population used was consistently multipotent, as expected for stem cells.

Chondrocyte Formation from OA BMSCs

In our initial experiments we cultured OA BMSCs with TGF-_{—}3 in high-density pellets. Under these conditions the pellets grew into cartilage-like nodules that were visible to the naked eye (FIG. 1A). At the histological level these nodules contained a large number of cells as well as an extracellular matrix that stained consistently for proteoglycan, although this was largely in a pericellular location (FIG. 1B). Similarly there was staining for collagens II and I throughout the pellets and this was most intense around the cells (FIG. 1C).

### Cartilage Tissue Engineering Using OA BMSCs

We were able successfully to engineer three-dimensional cartilage using a carefully ordered sequence of signals, as described in Materials and Methods. First, the OA BMSCs were expanded in 10% FCS and 1 ng/ml FGF-2. Second, the expanded cells were seeded onto PGA scaffolds that had been pre-coated with fibronectin. Third, they were cultured on a gently rotating platform for 1 week with 10 ng/ml TGF-β3 in differentiation medium. Fourth, they were cultured for a further 4 weeks on the rotating platform in differentiation medium with 50 μg/ml insulin as well as 10 ng/ml TGF-β3. Under these carefully defined conditions we were able to generate a white, shiny tissue that resembled hyaline cartilage at a macroscopic level (FIG. 2A). On histological analysis these cartilage constructs were found to contain rounded cells and an extracellular matrix that stained extensively for proteoglycan (FIG. 2B), moderately for type II collagen and weakly for type I collagen (FIG. 2C). Negative and positive controls for the immunostaining are shown in FIG. 2D.

### Biochemical Analysis of Engineered Cartilage

We undertook an extensive quantitative analysis of the engineered cartilage using a series of well-validated and specific assays (34). Micromass pellet cultures were also analysed, for comparison. Despite the sensitive nature of our assays (34), we had to use a minimum of 500,000 cells per micromass pellet and combine three such pellets at the end of culture in order to generate enough extracellular matrix for quantitative analysis. For tissue engineering we were able to use as few as 300,000 cells per scaffold and analyse one sample at a time. Cultures were maintained in TGF-β3 for up to 35 days prior to analysis. However in the case of pellets there was evidence of some loss of matrix beyond 21 days (not shown) and so cultures were stopped at this optimal time point. The dry weight of pellets and engineered tissue was calculated after freeze drying. In the case of engineered tissue the weight of any remaining PGA scaffold was determined after enzymic digestion of the extracellular matrix and this was subtracted from the total dry weight. On this basis we determined that the extracellular matrix of engineered tissue was at least 5 times that of pellet cultures and this difference was significant (Table 1). Furthermore the engineered cartilage contained significantly more proteoglycan and type II collagen than micromass pellet cultures. There was also a slightly higher type I collagen content, although this was still less than 10% of the type II collagen content in engineered cartilage.

We have previously found that the best cartilage tissue engineering could be achieved using bovine nasal chondrocytes (BNCs) (33). We therefore compared the results of cartilage engineering from OA BMSCs and from BNCs. There was no significant difference in the content of type II collagen, type I collagen or proteoglycan between the two cell types (FIG. 3), indicating that chondrocytes derived from OA BMSCs are as effective as the BNCs.

TABLE 1
Biochemical analysis of cartilage extracellular matrix

| | Pellet Culture | ⁴Tissue Engineering |
|---|---|---|
| Analytic Parameter | n = 10 | n = 15 |
| ¹Number of Cells | 500,000 | 300,000 |
| Seeded | | |
| Dry Weight | 0.23 ± 0.03 | 1.25 ± 0.77*** |
| Mean ± SEM | | |
| (mg) | | |
| ²[Type II Collagen] | 0.57 ± 0.07 | 17.21 ± 2.88*** |
| Mean ± SEM | | |
| (% of Dry Weight | | |
| ³[Proteoglycan] | 1.14 ± 0.13 | 29.96 ± 3.45*** |
| Mean ± SEM | | |
| (% of Dry Weight) | | |
| ²[Type I Collagen] | 0.26 ± 0.11 | 1.76 ± 0.25*** |
| Mean ± SEM | | |
| (% of Dry Weight) | | |

¹Minimum number of cells required for accurate quantification using biochemical assays
²Specific immunoassay after selective extraction of peptide epitopes
³Dimethylmethylene blue colorimetric assay of glucosaminoclycans
⁴Mann-Whitney U-test: ***p < 0.0001 v. pellet culture

### Inhibition of Hypertrophy by PTHrP.

In preliminary experiments we observed that OA BMSCs cultured with TGF-β3 in micromass pellets had an increased expression of type X collagen, indicating that these cells were likely to generate hypertrophic rather than hyaline cartilage. We therefore investigated the potential of inhibiting this hypertrophy using PTHrP, which has been shown to prevent maturation of pre-hypertrophic chondrocytes in the growthplate. BMSCs cultured in monolayer without TGF-β3 expressed very little type X collagen, however its expression was significantly upregulated when the same cells were used to engineer cartilage in a TGF-β3-driven system (FIG. 4A). PTHrP suppressed this upregulation of type X collagen mRNA in a significant and dose-dependent manner (FIG. 4A). Similarly, PTHrP at 10 μM significantly reduced the alkaline phosphatase content of cells in our engineered cartilage (FIG. 4B).

Having demonstrated that PTHrP can suppress early markers of hypertrophy, we considered it important to demonstrate that there was no reduction in the quality of engineered cartilage in the PTHrP cultures. It had no effect on mRNA or protein for the cartilage-specific type II collagen (FIG. 5A). Type I collagen, which is normally absent from hyaline cartilage, was further reduced from its already low level in these cartilage constructs, at both the mRNA and protein level (FIG. 5B). This led to a 4-fold, significant improvement in the ratio of collagen II to collagen I (FIG. 5C), with no effect on the proteoglycan content (FIG. 5D).

### Discussion

We have demonstrated for the first time the feasibility of tissue engineering hyaline cartilage from OA BMSCs. Biochemically, the cartilage quality was comparable to that achieved using the best available cell source, namely bovine nasal chondrocytes (33). Furthermore, we have shown that the tendency of BMSCs to become hypertrophic can be down regulated using PTHrP. These findings suggest that it will be feasible to develop a method of cartilage repair for OA patients using their own bone marrow cells to generate three-dimensional cartilage implants.

This study has utilised stem cells derived from patients with hip OA undergoing arthroplasty at a large orthopaedic referral centre in Bristol. As such this patient group is likely to be typical of those patients who might benefit from cartilage implantation. We have demonstrated that despite their poor capacity to produce any extracellular matrix in micromass pellet cultures, these cells can be directed to produce cartilage through the use of a series of specific molecular signals, applied in appropriate order. First, the adherent mesenchymal cells must be driven to proliferate so that their cell number can be expanded within a reasonable time-frame. Murphy et al (28) have found that the proliferation rate of OA BMSCs cultured in 10% serum was reduced compared to that of control cells. In the present study we used 1 ng/ml FGF-2 in addition to serum. This growth factor has been previously shown to enhance the proliferation of normal BMSCs (30, 31). More recently, we have found that OA BMSC proliferation is enhanced by FGF-2 and that the mechanism is dependent on the stem cell nucleolar protein, nucleostemin (29). This suggests that the reduced proliferative capacity identified by Murphy et al can be overcome by using this growth factor. The second molecular signal we used was fibronectin, coated onto the PG scaffolds in order to enhance adhesion of the OA BMSCs. Fibronectin has been shown previously to promote the adhesion of normal mesenchymal cells (38) and in our hands the same is true for those derived from OA patients. The third molecular signal was TGF-β3. There is extensive evidence to show that growth factors of the TGF superfamily promote chondrogenesis in micromass pellet cultures of normal human or animal BMSCs (22-24, 27) and we have now shown that it is effective at driving chondrogenesis from OA BMSCs. The fourth signal was 50 μg/ml insulin, which was added to the tissue engineering cultures one week after the start of differentiation by TGF-β, to promote the formation of extracellular matrix by the differentiated cells (39). Finally, we investigated the use of PTHrP as a fifth signal. Previous studies (22, 26) have shown that the TGF-βs promote the formation of hypertrophic chondrocytes, as shown by upregulation of type X collagen mRNA. PTHrP is known to down-regulate the maturation of pre-hypertrophic chondrocytes in the growth plate (40) and therefore we investigated its effects in our tissue engineering cultures. Not only did it down-regulate the early hypertrophic markers, it also enhanced the biochemical quality of our extracellular matrix as shown by the down-regulation of type I collagen whilst type II collagen and proteoglycan were maintained.

We have been able to generate cartilage that is of the highest quality compared to that generated using BNCs, which we have previously shown to be excellent chondrocytes for cartilage engineering (33). However this engineered cartilage has a lower collagen content than natural tissue, a feature that is true of all cartilage engineered in vitro (18, 19, 33, 39, 41). Whilst there is growing evidence that even very immature cartilage constructs can mature into natural hyaline cartilage once implanted within the joint (42, 43), it would nevertheless be preferable to engineer fully matured tissue in vitro prior to implantation. It is at present unclear if such maturation can be achieved in vitro.

Our findings support and develop the work of others who have shown that normal BMSCs can be used to generate chondrocytes (20, 22-25, 27). Murphy et al (28) described the poor capacity of OA BMSCs to proliferate and to form chondrocytes in micromass pellet cultures. We have overcome this reduced potential of the OA-derived cells using the range of molecular signals described above combined with the use of a PGA scaffold. Li et al (25) described the importance of using scaffolds to generate cartilage with enough volume and mass to be implanted. However in their studies the histological data suggested that the cartilage quality was no better than that achieved using micromass pellet cultures. The reason that we have successfully generated constructs of enhanced quality is presumably because of the use of the specific molecular signals together with a biomaterial scaffold.

Our conclusion that OA BMSCs can be used to generate relatively mature cartilage implants opens up the possibility of developing a cartilage therapy utilising autologous stem cells. The use of autologous cells has several advantages. It avoids the risk of immune rejection or the need for immunosuppression that would be required for donor cells. It also avoids the risk of disease transmission from donor to patient. There is currently intensive research into the use of embryonic stem cells to generate chondrocytes (21, 44) as well as other cells. Whilst of scientific importance, it is currently unclear if embryonic cell lines will ever be used in the clinical setting. Apart from the ethical concerns some patients would have, there is an inherent risk of teratoma formation as well as the potential for immune rejection that must be managed (21).

Autologous stem cells provide an attractive option for patients and clinicians. However it must also be recognized that autologous therapies are expensive, requiring growth of cells and tissue over several weeks in specialized ultraclean-rooms. Therefore it will be important to develop our tissue engineering protocol so that it can be undertaken in the shortest possible time in order to reduce costs. We also need to develop methods of attaching the cartilage implants to the subchondral bone and of promoting integration of the implant with surrounding tissue. Despite these challenges, our findings reported here represent a step forward in the development of an autologous cartilage replacement therapy.

### REFERENCES

1. Poole A R. Cartilage in health and disease. In: Koopman W J, editor. Arthritis and Allied Conditions: A Textbook of Rheumatology. Baltimore: Williams & Wilkins; 1997. p. 255-308.
2. Hollander A P, Heathfield T F, Webber C, Iwata Y, Bourne R, Rorabeck C, et al. Increased damage to type II collagen in osteoarthritic articular cartilage detected by a new immunoassay. J. Clin. Invest. 1994; 93:1722-1732.
3. Hollander A P, Pidoux I, Reiner A, Rorabeck C, Bourne R, Poole A R. Damage to type II collagen in aging and osteoarthritis starts at the articular surface, originates around chondrocytes, and extends into the cartilage with progressive degeneration. Journal of Clinical Investigation 1995; 96:2859-2869.
4. Caterson B, Flannery C R, Hughes C E, Little C B. Mechanisms of proteoglycan metabolism that lead to cartilage destruction in the pathogenesis of arthritis. Drugs Today (Barc) 1999; 35(4-5):397-402.
5. Hunziker E B. Articular cartilage repair: are the intrinsic biological constraints undermining this process insuperable? Osteoarthritis Cartilage 1999; 7(1):15-28.
6. Steadman J R, Rodkey W G, Rodrigo J J. Microfracture: surgical technique and rehabilitation to treat chondral defects. Clin Orthop 2001(391 Suppl):S362-9.
7. Cawston T E. Blocking cartilage destruction with metalloproteinase inhibitors: a valid therapeutic target? Annals of the Rheumatic Diseases 1993; 52:769-770.
8. Greenwald R A. Thirty-six years in the clinic without an MMP inhibitor. What hath collagenase wrought? Ann N Y Acad Sci 1999; 878:413-9.
9. Close D R. Matrix metalloproteinase inhibitors in rheumatic diseases. Ann Rheum Dis 2001; 60 Suppl 3:iii62-7.
10. Smith G D, Knutsen G, Richardson J B. A clinical review of cartilage repair techniques. J Bone Joint Surg Br 2005; 87(4):445-9.
11. Hangody L, Feczko P, Bartha L, Bodo G, Kish G. Mosaicplasty for the treatment of articular defects of the knee and ankle. Clin Orthop 2001(391 Suppl):S328-36.
12. Peterson L, Minas T, Brittberg M, Nilsson A, Sjogren-Jansson E, Lindahl A. Two- to 9-year outcome after autologous chondrocyte transplantation of the knee. Clin Orthop 2000(374):212-34.
13. Minas T, Peterson L. Advanced techniques in autologous chondrocyte transplantation. Clin Sports Med 1999; 18(1):13-44, v-vi.
14. Trattnig S, Ba-Ssalamah A, Pinker K, Plank C, Vecsei V, Marlovits S. Matrix-based autologous chondrocyte implantation for cartilage repair: noninvasive monitoring by high-resolution magnetic resonance imaging. Magn Reson Imaging 2005; 23(7):779-87.
15. Pritzker K P, Gay S, Jimenez S A, Ostergaard K, Pelletier J P, Revell P A, et al. Osteoarthritis cartilage histopathology: grading and staging. Osteoarthritis Cartilage 2006; 14(1):13-29.
16. Langer R. Vacanti J P. Tissue engineering. Science 1993; 260(5110):920-6.
17. Langer R. 1994 Whitaker lecture: polymers for drug delivery and tissue engineering. Annals of Biomedical Engineering 1995; 23:101-111.
18. Freed L E, Hollander A P, Martin I, Barry J R, Langer R, Vunjak-Novakovic G. Chondrogenesis in a cell-polymer bioreactor system. Experimental Cell Research 1998; 240:58-65.
19. Riesle J, Hollander A P, Langer R, Freed L E, Vunjak-Novakovic G. Collagen in tissue engineered cartilage; types, structure and crosslinks. Journal of Cellular Biochemistry 1998; 71:313-327.
20. Raghunath J, Salacinski H J, Sales K M, Butler P E, Seifalian A M. Advancing cartilage tissue engineering: the application of stem cell technology. Curr Opin Biotechnol 2005; 16(5):503-9.
21. Vats A, Bielby R C, Tolley N S, Nerem R, Polak J M. Stem cells. Lancet 2005; 366(9485):592-602.
22. Martin I, Shastri V P, Padera R F, Yang J, Mackay A J, Langer R, et al. Selective differentiation of mammalian bone marrow stromal cells cultured on three dimensional polymer foams. J Biomed Mater Res 2001; 55(2):229-35.
23. Yoo J U, Barthel T S, Nishimura K, Solchaga L, Caplan A I, Goldberg V M, et al. The chondrogenic potential of human bone-marrow-derived mesenchymal progenitor cells. J Bone Joint Surg Am 1998; 80(12):1745-57.
24. Johnstone B, Hering T M, Caplan A I, Goldberg V M, Yoo J U. In vitro chondrogenesis of bone marrow-derived mesenchymal progenitor cells. Exp Cell Res 1998; 238(1):265-72.
25. Li W J, Tuli R, Okafor C, Derfoul A, Danielson K G, Hall D J, et al. A three-dimensional nanofibrous scaffold for cartilage tissue engineering using human mesenchymal stem cells. Biomaterials 2005; 26(6):599-609.
26. Cheng H, Jiang W, Phillips F M, Haydon R C, Peng Y, Zhou L, et al. Osteogenic activity of the fourteen types of human bone morphogenetic proteins (BMPs). J Bone Joint Surg Am 2003; 85-A(8):1544-52.
27. Majumdar M K, Banks V, Peluso D P, Morris E A. Isolation, characterization, and chondrogenic potential of human bone marrow-derived multipotential stromal cells. J Cell Physiol 2000; 185(1):98-106.
28. Murphy J M, Dixon K, Beck S, Fabian D, Feldman A, Barry F. Reduced chondrogenic and adipogenic activity of mesenchymal stem cells from patients with advanced osteoarthritis. Arthritis Rheum 2002; 46(3):704-13.
29. Kafienah W, Mistry S, Williams C, Hollander A P. Nucleostemin is a marker of proliferating stromal stem cells in adult human bone marrow. Stem Cells 2006; In Press.
30. Martin I, Muraglia A, Campanile G, Cancedda R, Quarto R. Fibroblast growth factor-2 supports ex vivo expansion and maintenance of osteogenic precursors from human bone marrow. Endocrinology 1997; 138(10):4456-62.
31. Solchaga L A, Penick K, Porter J D, Goldberg V M, Caplan A I, Welter J F. FGF-2 enhances the mitotic and chondrogenic potentials of human adult bone marrow-derived mesenchymal stem cells. J Cell Physiol 2005; 203(2):398-409.
32. Freed L E, Vunjak-Novakovic G, Biron R J, Eagles D B, Lesnoy D C, Barlow S K, et al. Biodegradable polymer scaffolds for tissue engineering. Bio/Technology 1994; 12:689-693.
33. Kafienah W, Jakob M, Demarteau O, Frazer A, Barker M D, Martin I, et al. Three dimensional tissue engineering of hyaline cartilage: comparison of adult nasal and articular chondrocytes. Tissue Engineering 2002; 8:817-826.
34. Dickinson S C, Sims T J, Pittarello L, Soranzo C, Pavesio A, Hollander A P. Quantitative outcome measures of cartilage repair in patients treated by tissue engineering. Tissue Eng 2005; 11 (1-2):277-87.
35. Handley C J, Buttle D J. Assay of proteoglycan degradation. Methods Enzymol. 1995; 248:47-58.
36. Yarram S J, Tasman C, Gidley J, Clare M, Sandy J R, Mansell J P. Epidermal growth factor and calcitriol synergistically induce osteoblast maturation. Mol Cell Endocrinol 2004; 220(1-2):9-20.
37. Raff T, van der Giet M, Endemann D, Wiederholt T, Paul M. Design and testing of betaactin primers for RT-PCR that do not co-amplify processed pseudogenes. Biotechniques 1997; 23(3):456-60.
38. Cool S M, Nurcombe V. Substrate induction of osteogenesis from marrow-derived, mesenchymal precursors. Stem Cells Dev 2005; 14(6):632-42.
39. Blunk T, Sieminski A L, Gooch K J, Courter D L, Hollander A P, Nahir A M, et al. Differential effects of growth factors on tissue-engineered cartilage Tissue Eng 2002; 8(1):73-84.
40. Stott N S, Chuong C M. Dual action of sonic hedgehog on chondrocyte hypertrophy: retrovirus mediated ectopic sonic hedgehog expression in limb bud micromass culture induces novel cartilage nodules that are positive for alkaline phosphatase and type X collagen. J Cell Sci 1997; 110 (Pt 21):2691-701.
41. Marijnissen W J, van Osch G J, Aigner J, Verwoerd-Verhoef H L, Verhaar J A. Tissue engineered cartilage using serially passaged articular chondrocytes. Chondrocytes in alginate, combined in vivo with a synthetic (E210) or biologic biodegradable carrier (DBM). Biomaterials 2000; 21(6):571-80.
42. Hollander A P, Dickinson S C, Sims T J, Soranzo C, Pavesio A. Quantitative analysis of repair tissue biopsies following chondrocyte implantation. Novartis Found Symp 2003; 249:218-29.
43. Hollander A P, Dickinson S C, Sims T J, Brun P, Cortivo R, Kon E, et al. Maturation of engineered cartilage implanted in injured and osteoarthritic human knees. Tissue Eng 2006; In Press.
44. Vats A, Bielby R C, Tolley N, Dickinson S C, Boccacini A R, Hollander A P, et al. Chondrogenic differentiation of human embryonic stem cells: the effect of the microenvironment. Tissue Engineering 2006; In Press.
45. Kronenberg H M 2003 Developmental regulation of the growth plate. Nature 423(6937):332-6.
46. Chung Ul, Lanske B, Lee K, Li E, Kronenberg H 1998 The parathyroid hormone/parathyroid hormone-related peptide receptor coordinates endochondral bone development by directly controlling chondrocyte differentiation. Proc Natl Acad Sci USA 95(22):13030-5.
47. Lee K, Lanske B, Karaplis A C, Deeds J D, Kohno H, Nissenson R A, Kronenberg H M, Segre G V 1996 Parathyroid hormone-related peptide delays terminal differentiation of chondrocytes during endochondral bone development. Endocrinology 137(11):5109-18.
48. Vortkamp A, Lee K, Lanske B, Segre G V, Kronenberg H M, Tabin C J 1996 Regulation of rate of cartilage differentiation by Indian hedgehog and PTH-related protein. Science 273(5275):613-22.
49. Castagnola P, Dozin B, Moro G, Cancedda R 1988 Changes in the expression of collagen genes show two stages in chondrocyte differentiation in vitro. J Cell Biol 106(2):461-7.
50. Kirsch T, Nah H D, Shapiro 1M, Pacifici M 1997 Regulated production of mineralization-competent matrix vesicles in hypertrophic chondrocytes. J Cell Biol 137(5):1149-60.
51. Amling M, Neff L, Tanaka S, Inoue D, Kuida K, Weir E, Philbrick W M, Broadus A E, Baron R 1997 Bcl-2 lies downstream of parathyroid hormone-related peptide in a signaling pathway that regulates chondrocyte maturation during skeletal development. J Cell Biol 136(1):205-13.
52. Roach H I 1992 Trans-differentiation of hypertrophic chondrocytes into cells capable of producing a mineralized bone matrix. Bone Miner 19(1):1-20.
53. Henderson J E, He B., Goltzman D, Karaplis A C 1996 Constitutive expression of parathyroid hormone-related peptide (PTHrP) stimulates growth and inhibits differentiation of CFK2 chondrocytes. J Cell Physiol 169(1):33-41.
54. Iwasaki M, Jikko A, Le A X 1999 Age-dependent effects of hedgehog protein on chondrocytes. J Bone Joint Surg Br 81(6):1076-82.
55. Lanske B, Karaplis A C, Lee K, Luz A, Vortkamp A, Pirro A, Karperien M, Defize L H, Ho C, Mulligan R C, Abou-Samra A B, Juppner H, Segre G V, Kronenberg H M 1996 PTH/PTHrP receptor in early development and Indian hedgehog-regulated bone growth. Science 273(5275):663-6.
56. Weir E C, Philbrick W M, Amling M, Neff L A, Baron R, Broadus A E 1996 Targeted overexpression of. parathyroid hormone-related peptide in chondrocytes causes chondrodysplasia and delayed endochondral bone formation. Proc Natl Acad Sci USA 93(19):10240-5.
57. Schipani E, Lanske B. Hunzelman J. Luz A, Kovacs C S, Lee K, Pirro A, Kronenberg H M, Juppner H 1997 Targeted expression of constitutively active receptors for parathyroid hormone and parathyroid hormone-related peptide delays endochondral bone formation and rescues mice that lack parathyroid hormone-related peptide. Proc Natl Acad Sci USA 94(25): 13689-94.
58. Terkeltaub R A, Johnson K, Rohnow D, Goomer R, Burton D, Deftos L J 1998 Bone morphogenetic proteins and bFGF exert opposing regulatory effects on PTHrP expression and inorganic pyrophosphate elaboration in immortalized murine endochondral hypertrophic chondrocytes (MCT cells). J Bone Miner Res 13(6):931-41.
59. Caplan A I 1991 Mesenchymal stem cells. J Orthop Res 9(5):641-50.
60. Caplan A I, Bruder S P 2001 Mesenchymal stem cells: building blocks for molecular medicine in the 21st century. Trends Mol Med 7(6):259-64.
61. Jiang Y, Jahagirdar B N, Reinhardt R L, Schwartz R E, Keene C D, Ortiz-Gonzalez X R, Reyes M, Lenvik T, Lund T, Blackstad M, Du J, Aldrich S, Lisberg A, Low W C, Largaespada D A, Verfaillie C M 2002 Pluripotency of mesenchymal stem cells derived from adult marrow. Nature 418(6893):41-9.
62. Suva D, Garavaglia G, Menetrey J, Chapuis B, Hoffmeyer P, Bernheim L, Kindler V 2004 Non-hematopoietic human bone marrow contains long-lasting, pluripotential mesenchymal stem cells. J Cell Physiol 198(1):110-8.
63. D'lppolito G, Diabira S, Howard G A, Menei P, Roos B A, Schiller P C 2004 Marrow-isolated adult multilineage inducible (MIAMI) cells, a unique population of postnatal young and old human cells with extensive expansion and differentiation potential. J Cell Sci 117(Pt 14):2971-81.
64. Fibbe W E 2002 Mesenchymal stem cells. A potential source for skeletal repair. Ann Rheum Dis 61 Suppl 2:ii29-31.
65. Shea C M, Edgar C M, Einhorn T A, Gerstenfeld L C 2003 BMP treatment of C3H10T1/2 mesenchymal stem cells induces both chondrogenesis and osteogenesis. J Cell Biochem 90(6):1112-27.
66. Mastrogiacomo M, Cancedda R, Quarto R 2001 Effect of different growth factors on the chondrogenic. potential of human bone marrow stromal cells. Osteoarthritis Cartilage 9 Suppl A:S36-40.
67. Winter A, Breit S, Parsch D, Benz K, Steck E, Hauner H. Weber R M, Ewerbeck V, Richter W 2003 Cartilage-like gene expression in differentiated human stem cell spheroids: a comparison of bone marrow-derived and adipose tissue-derived stromal cells. Arthritis Rheum 48(2):418-29.
68. Steinert A, Weber M, Dimmler A, Julius C, Schutze N, Noth U, Cramer H, Eulert J, Zimmermann U, Hendrich C 2003 Chondrogenic differentiation of mesenchymal progenitor cells encapsulated in ultrahigh-viscosity alginate. J Orthop Res 21(6):1090-7.

1. A method for engineering three dimensional cartilage constructs from chondrogenic cells, said method comprising a step of treating the chondrogenic cells or immature constructs with PTHrP to regulate hypertrophy.

2. The method according to claim 1, which comprises incubating chondrogenic cells in the presence of PTHrP during in vitro maturation of the cartilage constructs.

3. The method according to claim 1, wherein the chondrogenic cells are seeded onto a bioactive scaffold capable of controlled release of PTHrP in situ following implantation of the engineered cartilage construct.

4. The method according to claim 1, which comprises delivering PTHrP to the chondrogenic cells or immature construct, after implantation of the construct in a patient, by injection into the joint, systemic injection or oral administration.

5. The method according to any of claims 1 to 4, wherein the chondrogenic cells are bone marrow stromal cells.

6. Three dimensional cartilage produced by the method of any of claims 1 to 5.

7. An engineered cartilage construct comprising chondrogenic cells and a bioactive scaffold capable of controlled release of PTHrP.

8. Use of PTHrP in the manufacture of a medicament for the regulation of hypertrophy in engineered cartilage.

9. Use of PTHrP in the manufacture of engineered cartilage for the repair of damaged cartilage.

10. Use according to claim 9, wherein the cartilage damage is the result of osteoarthritis.

11. A method for the treatment of osteoarthritis which comprises the step of administering to a patient in need thereof an effective amount of PTHrP, wherein hypertrophy of osteoarthritic chondrocytes is reversed or delayed.

12. A method of screening compounds for the ability to inhibit hypertrophy in chondrogenic cells, which comprises incubating a test compound with chondrogenic or chondrogenic progenitor cells and determining the production of type X collagen by the cells relative to control cells.
